(19) United States
(12) Patent Application Publication
(10) Pub. No.: US 2022/0080029 A1
(43) Pub. Date: Mar. 17, 2022

(54) COMPOSITION COMPRISING SERICIN FOR TREATING, PREVENTING, OR ALLEVIATING FATTY LIVER, AND METHOD OF PREPARING SAME

(71) Applicant: NI&Pharm Inc., Seoul (KR)

(72) Inventor: Hyuk Jin KWON, Gyeonggi-do (KR)

(73) Assignee: NI&Pharm Inc., Seoul (KR)

(21) Appl. No.: 17/420,130

(22) PCT Filed: Dec. 23, 2019
(86) PCT No.: PCT/KR2019/018345

§ 371 Date: Jun. 30, 2021

(30) Foreign Application Priority Data
Dec. 31, 2018 (KR) 10-2018-0173648
Dec. 19, 2019 (KR) 10-2019-0171115

(51) Int. Cl. A61K 38/17; A61P 1/16; A23L 33/18

(52) U.S. Cl. CPC A61K 38/1767; A23L 33/18; A61P 1/16

(57) ABSTRACT

A sericin-containing composition for treatment, prevention, or alleviation of fatty liver and a method of preparing the same.

### TECHNICAL FIELD

The present invention relates to a sericin-containing composition for treatment, prevention, or alleviation of fatty liver disease and a method of preparing the same.

### BACKGROUND ART

The death rate from liver disease in Korea is very high (23.5 cases per 100,000 population) and liver disease is the leading cause of death among middle-aged Koreans. Specifically, liver disease is the first cause of death among adults aged 40 to 49 (41.1 per 100,000), the second cause of death among adults aged 50 to 59 (72.4 per 100,000), and the third cause of death among adults aged 30 to 39 (10 per 100,000).

Among liver diseases, fatty liver is a pathological condition in which triglyceride, which does not exist in normal cells, abnormally accumulates in liver cells. Normal liver is composed of about 5% of fatty tissue, which includes triglyceride, fatty acid, phospholipid, cholesterol, and cholesterol ester as major constituents. Once fatty liver occurs, most of the constituents are converted into triglyceride. When the proportion of triglyceride in the liver is 5 wt % or more, diagnosis of fatty liver is made. When a lump of fat in the liver becomes larger with increasing severity of fatty liver, major constituents of liver cells, including nuclei, are shifted to one side, causing malfunction of the liver cells. In addition, the liver cells expanding due to accumulation of fat therein press microvessels and lymph glands located between the liver cells, causing poor circulation of blood and lymph. As a result, proper supply of oxygen and nutrients to the liver cells cannot be achieved, causing deterioration in liver functions.

Non-alcoholic fatty liver disease (NAFLD) is not liver injury due to alcohol and is defined as a condition in which, based on the total weight of the liver, 5 wt % or more of fatty acid accumulates in the form of triglyceride within mesenchymal cells of the liver. Pathologically, NAFLD is classified as hepatic steatosis accompanied by simple steatosis and inflammation. However, when left for a long time, NAFLD can develop into severe liver disease, such as hepatitis, liver fibrosis, and cirrhosis. In Korea, there is rising incidence of non-alcoholic fatty liver disease due to change in lifestyle.

Sericin is a scleroprotein and constitutes silkworm cocoon fiber together with fibroin. Generally, sericin is known to have moisturizing effects, antioxidant activity, and UV protection functions, and is mainly used in cosmetics.

### DISCLOSURE

### Technical Problem

The present invention has been conceived in view of the aforementioned technical background and it is one object of the present invention to provide a composition which includes sericin, which is a silk protein derived from silkworm cocoons, as an active principle in treatment, prevention, or alleviation of fatty liver disease.

It is another object of the present invention to provide a method of preparing the composition set forth above.

### Technical Solution

One aspect of the present invention relates to a sericin-containing composition for treatment, prevention, or alleviation of fatty liver disease.

Generally, sericin is removed from silk by degumming, whereby the silk can have unique gloss and texture. Despite accounting for about 25% of the total silk protein, the sericin is mostly discarded after degumming. If degumming waste liquor containing the sericin is released into rivers, the rivers eventually undergo eutrophication, causing environmental pollution.

Therefore, various methods have been studied to recover and recycle the sericin, which would otherwise be discarded. Examining amino acid composition of the sericin, serine accounts for approximately 30% of the total amino acid content and the content of hydrophilic amino acids is high. In particular, since the amino acid composition of the sericin is similar to that of a natural moisturizing factor (NMF) of the human body, the sericin is known as a material that is highly effective in skin moisturizing. In addition, sericin is known to be effective in anti-oxidation, whitening through inhibition of lipid peroxidation and tyrosinase activity, suppression of skin cancer, and the like.

The inventors of the present invention found that sericin, normally considered as a waste material, is effective in treating, preventing or alleviating fatty liver disease.

Sericin useable in the present invention may be obtained by extracting only sericin components from silk using an aqueous solution of soap, acid, or alkali. For example, the sericin may be extracted from silkworm cocoons at high temperature or at high pressure using only water, or may be prepared by removing impurities from a sericin solution through dialysis or the like, wherein the sericin solution is obtained by treating silkworm cocoons with an aqueous solution of sodium carbonate, followed by heating and filtration. Here, the sericin solution with impurities removed therefrom may be used as prepared or may be freeze-dried into powder form. Here, the aqueous solution of sodium carbonate may have a concentration of 0.001 M to 2M, specifically 0.002 M to 1 M, the heating temperature may range from 70° C. to 130° C., specifically 80° C. to 120° C., and the heating time may range from 5 minutes to 3 hours, specifically 30 minutes to 2 hours.

Alternatively, the sericin may be prepared by a synthesis method. Here, the synthesis method may be synthesis using microorganisms or polypeptide synthesis commonly used in the art.

The sericin may have a molecular weight distribution in a range of 200 Da to 400 kDa. Specifically, the sericin may have a molecular weight distribution having two main peaks. Here, the two main peaks may consist of a first main peak located between 1,000 Da and 1,700 Da and a second main peak located between 10 kDa and 30 kDa. More specifically, the sericin may have a weight average molecular weight of about 1,200 Da to about 1,600 Da, specifically about 1,300 Da to about 1,500 Da, more specifically about 1,427 Da, as determined from a molecular weight distribution curve which includes the first main peak appearing between 1,000 Da and 1,700 Da. In addition, the sericin may have a weight average molecular weight of about 15 kDa to about 20 kDa, specifically about 16 kDa to about 19 kDa, more specifically about 18 kDa, as determined from a molecular weight distribution curve which includes the second main peak appearing between 10 kDa and 30 kDa.

The sericin may be present in an amount of 0.01 wt % to 90 wt %, specifically 0.01 wt % to 70 wt %, based on the total weight of the composition.

As used herein, “fatty liver disease” refers to abnormal accumulation of fat within liver cells, specifically a pathological condition in which fat content of the liver is 3 wt % or more, specifically 5 wt % or more. Fatty liver disease is divided into alcoholic fatty liver disease due to heavy alcohol use and non-alcoholic fatty liver disease due to obesity, diabetes, hyperlipidemia, drugs and the like. The composition according to the present invention may be particularly effective in treatment or prevention of non-alcoholic fatty liver or steatohepatitis. Non-alcoholic fatty liver disease is caused by accumulation of fat within the liver due to reduction in fatty acid oxidation and increase in triglyceride biosynthesis which result from increase in migration of fatty acid from adipose tissue to the liver due to imbalance of energy consumption. If accumulation of fat becomes severe, fatty liver disease develops into steatohepatitis and eventually develops into liver fibrosis and liver cirrhosis as inflammatory reactions increase. Therefore, prevention of accumulation of fat in the liver is needed to prevent fatty liver disease, which is an initial pathological phase, from developing into liver cirrhosis. The composition according to the present invention is effective in treating, preventing or alleviating fatty liver disease, particularly non-alcoholic fatty liver or steatohepatitis. In addition, since sericin is the only silk peptide used as an active ingredient of the composition according to the present invention, the composition is more effective in treating or preventing fatty liver than a composition including other silk peptides or other hydrolyzed silk proteins.

The composition may further include another active ingredient helpful in treating, preventing, or alleviating fatty liver disease. Based on reports demonstrating a correlation between fatty liver disease and insulin resistance observed in diabetes and obesity, medicines for diabetes or medicines for high triglycerides, such as metformin, have been prescribed for treatment of fatty liver disease. Accordingly, in one embodiment, the other active ingredient may include a medicine for diabetes or a medicine for high triglycerides. Alternatively, the other active ingredient may include a silk fibroin peptide. Here, the silk fibroin peptide may include a peptide mixture prepared by degumming silkworm cocoons and removing sericin from the degummed silkworm cocoons, followed by degradation via protease or acid hydrolysis, wherein the peptide mixture may have a weight average molecular weight of 100 to 5,000, specifically 300 to 2,000.

Alternatively, the other active ingredient may include at least one HTR2A antagonist selected from the group of sarpogrelate, adatanserin, altanserin, AMDA, amperozide, asenapine, BL-1020, cinanserin, clozapine, deramciclane, fananserin, flibanserin, glemanserin, iferanserin, ketanserin, lidanserin, lubazodone, lumateperone, medifoxamine, mepiprazole, naftidrofuryl, volinanserin, spiperone, setoperone, ritanserin, risperidone, quetiapine, rauwolscine, pruvanserin, pipamperone, phenoxybenzamine, and olanzapine.

Alternatively, the other active ingredient may include lactic acid bacteria-fermented kelp extract, Hovenia dulcis podocarp extract, bellflower root extract, milk thistle extract, fermented turmeric, Rubus coreanus fruit extract, garcinia bark extract, conjugated linoleic acid, Camellia sinensis leaf extract, chitosan, green mate extract, green coffee bean extract, soybean embryo extract, lactoferrin, spirulina, phragmites rhizome extract, or Humulus japonicus extract.

The other active ingredient may be present in an amount of 0.01 wt % to 30 wt %, specifically 0.01 wt % to 25 wt %, more specifically 0.05 wt % to 20 wt %, based on the total weight of the composition.

The composition may further include a pharmaceutically or sitologically acceptable excipient commonly used in formulation, for example, fillers, an extender, a binder, a wetting agent, a flavoring agent, a preservative, a sweetener, a disintegrant, a surfactant, a carrier, or a diluent.

Examples of the excipient may include lactose, dextrose, sucrose, sorbitol, mannitol, xylitol, erythritol, maltitol, starch, acacia rubber, alginate, gelatin, calcium silicate, cellulose, methylcellulose, microcrystalline cellulose, water, talc, and magnesium stearate.

The composition may be administered by a variety of routes, for example, orally, intraperitoneally, rectally, intravenously, intra-arterially, intramuscularly, transdermally, subcutaneously, intrauterinely, durally or intracerebrovascularly. Specifically, the composition may be orally administered.

The composition may be used as food or medicine. In addition, the composition may be formulated into pills, tablets, capsules, powders, suspensions, granules, or liquids.

Dosage of the composition may vary depending on severity of a disease to be treated, conditions of each individual patient, route of administration, and dosage form. In general, daily dose of the sericin may range from 0.001 mg/kg to 2,000 mg/kg, specifically 0.1 mg/kg to 1,600 mg/kg, more specifically 0.5 mg/kg to 1,000 mg/kg.

Another aspect of the present invention relates to a method of preparing a composition for treatment, prevention or alleviation of fatty liver disease, which includes sericin and a pharmaceutically or sitologically acceptable excipient. The method includes applying the pharmaceutically or sitologically acceptable excipient to the sericin. Since details and content of each ingredient useable in this aspect are the same as in the above aspect, description thereof will be omitted.

A further aspect of the present invention relates to a method of treating, preventing or alleviating fatty liver disease, which includes administering a composition including sericin as an active principle to a subject in need of treatment, prevention or alleviation of fatty liver disease.

Daily dose of the sericin may range from 0.001 mg/kg to 2,000 mg/kg, specifically 0.1 mg/kg to 1,600 mg/kg, more specifically 0.5 mg/kg to 1,000 mg/kg. The composition may be administered by a variety of routes, for example, orally, intraperitoneally, rectally, intravenously, intra-arterially, intramuscularly, transdermally, subcutaneously, intrauterinely, durally or intracerebrovascularly. Specifically, the composition may be orally administered to the subject. Since details and content of each ingredient useable in this aspect are the same as in the above aspect, description thereof will be omitted.

### Advantageous Effects

The present invention provides a composition which has effects of reducing the level of liver inflammation in fatty liver disease, reducing fat content of the liver, and alleviating inflammation in the liver.

In addition, the composition according to the present invention is effective in treatment, prevention or alleviation of fatty liver disease.

### DESCRIPTION OF DRAWINGS

FIG. 1 is a graph depicting a molecular weight distribution of sericin prepared in one embodiment of the present invention, wherein two main peaks appear in the molecular weight distribution.

FIG. 2 is a graph depicting a region of the molecular weight distribution of FIG. 1 including a second main peak.

FIG. 3 is a graph depicting a region of the molecular weight distribution of FIG. 1 including a first main peak.

FIG. 4 shows respective graphs depicting differences in body weight and dietary intake between a control group and a sericin-treated group in one experimental example of the present invention.

FIG. 5 shows respective graphs depicting differences in liver weight and ratio of liver weight to body weight between a control group and a sericin-treated group in one experimental example of the present invention.

FIG. 6 shows respective graphs depicting differences in blood glucose level and AUC at 30 minutes upon oral glucose tolerance testing between a control group and a sericin-treated group in one experimental example of the present invention.

FIG. 7 shows images depicting degrees of hepatic fat accumulation and hepatic inflammatory infiltration of a control group and a sericin-treated group in one experimental example of the present invention.

FIG. 8 shows respective graphs showing differences in ALT, AST, TG, albumin, and cholesterol levels between a control group and a sericin-treated group in one experimental example of the present invention.

FIG. 9 is a diagram illustrating a plan for conducting experiments after establishing NASH-induced animal experimental models in one experimental example of the present invention.

### MODE FOR INVENTION

Next, the present invention will be described in more detail with reference to examples. However, it should be noted that these examples are provided for illustration only and should not be construed in any way as limiting the invention.

### EXAMPLE 1

Preparation of Sericin-Containing Composition

Sericin used in the present invention was prepared using cocoons of Bombyx mori. First, 50 kg of the cocoons and purified water (50 times the weight of the cocoons) were placed in a reactor, followed by boiling treatment for 6 hours. Then, the resulting product was subjected to filtration with a microfilter, followed by homogenization in the reactor for 30 minutes. A purified aqueous solution of protease (ratio of protease to substrate: 1:100) was introduced into the reactor, followed by hydrolysis at 55° C. for 24 hours. Then, the resulting reaction solution was heated to 95° C. for 30 minutes to remove enzymatic activity, followed by concentration under reduced pressure. Dextrin was added to and dissolved in the resulting concentrated solution such that sericin hydrolysate was present in an amount of 70% based on the total solid weight. Then, the resulting solution was subjected to sterilization at 95° C. for 30 minutes, followed by spray drying.

### EXAMPLE 2

Measurement of Molecular Weight

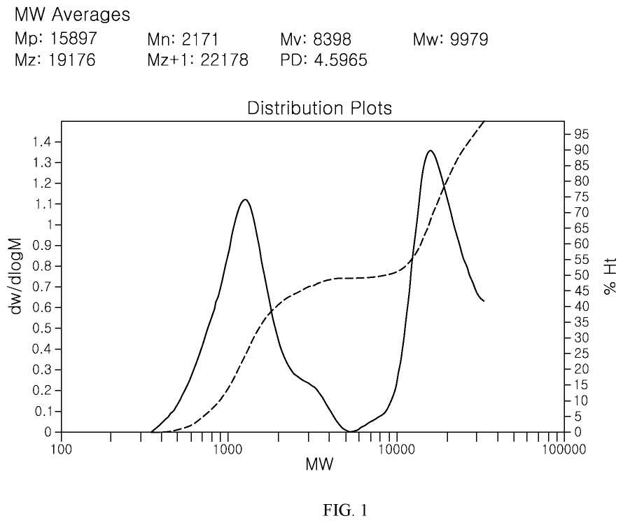

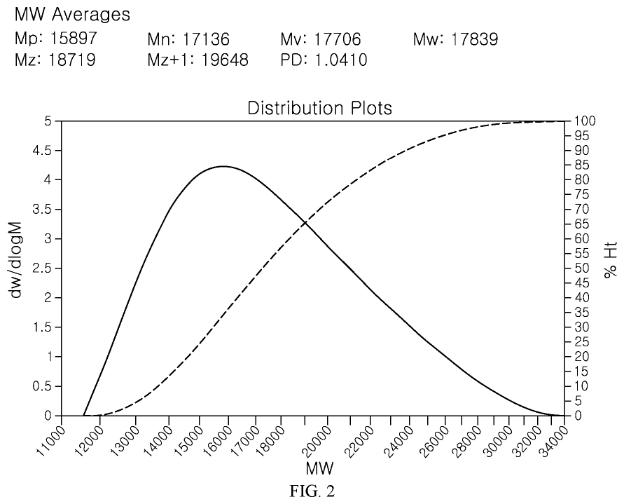

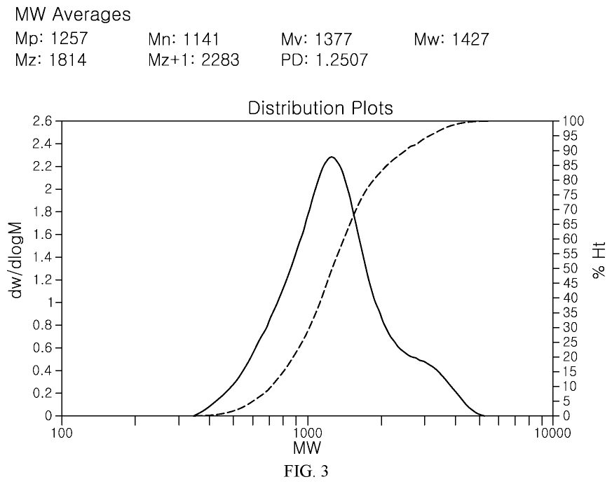

Molecular weight of the enzymatic sericin hydrolysate prepared in Example 1 was measured by gel permeation chromatography. Specifically, the enzymatic sericin hydrolysate was sampled using an HPLC instrument (Model 1100, Agilent Technologies) and the molecular weight of the sample was calculated using Agilent OpenLAB cirrus GPC software. Results of molecular weight measurement showed that the enzymatic sericin hydrolysate was a mixture that had a molecular weight distribution ranging from 200 Da to 400,000 Da and having two main peaks, wherein values of weight average molecular weight at first and second main peaks of a molecular weight distribution curve of the mixture were 1,427 Da and 17,839 Da, respectively (see FIG. 1 to FIG. 3).

3. Experimental Example

(1) Establishment of experimental model of fatty liver-induced animal

Eight week old C57BL/6 mice were purchased and used. At room temperature (22±2° C.) and 50% to 70% RH, the mice were allowed to freely ingest a high-fat diet (HFD) along with sufficient amounts of a general diet and water for 10 weeks to induce fatty liver in the mice. Here, ingredients that could affect experimental results were removed from the diets. Then, the non-alcoholic steatohepatitis (NASH)-induced animal experimental models were randomly divided into two groups. One group (control group, NASH)) was orally administered saline for 11 weeks, and the other group (NASH+S) was orally administered the sericin-containing composition prepared in Example 1 at a dose of 1,600 mg/kg for 11 weeks.

(2) Body Weight and Dietary Intake

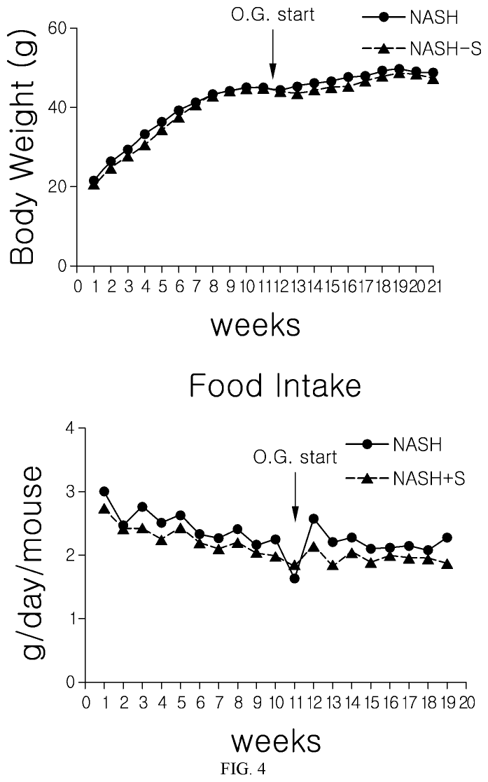

Body weight and dietary intake were measured on each mouse of the control group and the sericin-treated group at the beginning of each week. Results are shown in FIG. 4. There was no significant difference in body weight or dietary intake between the control group and the sericin-treated group throughout the experiment period.

(3) Liver Weight

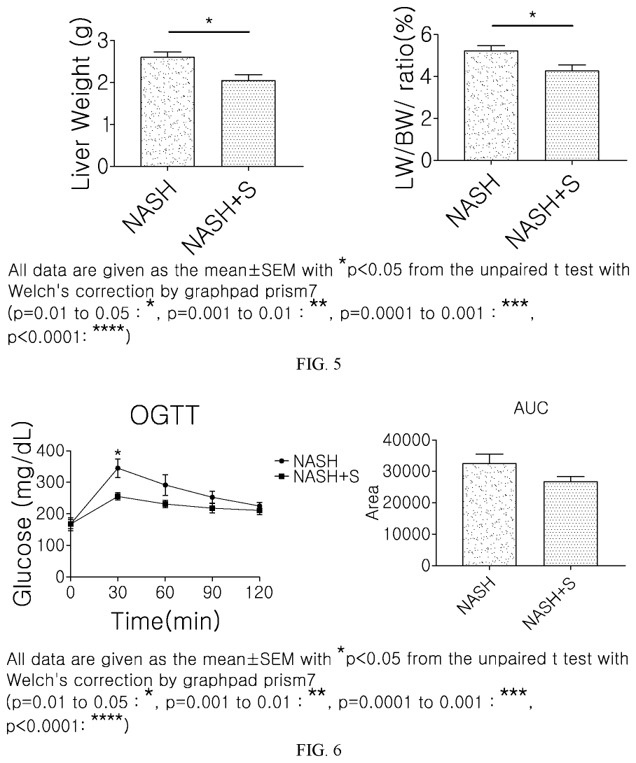

After completing the above experiment on each mouse of the control group and the sericin-treated group, an anesthetic solution obtained by diluting 50 mg/kg of zoletil and 50 mg/kg of Rompun in saline in a ratio of 1:1:2 was intraperitoneally injected into each mouse to induce anesthesia, followed by extraction of liver tissue, and then measurement of the weight of liver and the ratio of liver weight to body weight was performed along with staining assay of liver cells. Results are shown in FIG. 5. The weight of liver and the ratio of liver weight to body weight were significantly lower in the sericin-treated group than in the control group.

(4) Insulin Resistance

After completing the above experiment on each mouse of the control group and the sericin-treated group and before extraction of liver tissue, an insulin resistance test was conducted. Results are shown in FIG. 6. In an oral glucose tolerance test, the sericin-treated group had lower blood glucose levels than the control group, and, especially, showed significant reduction in blood glucose level at 30 minutes, as compared with the control group.

In addition, the sericin-treated group had lower AUC levels than the control group. Therefore, it can be seen that treatment with sericin can increase insulin resistance, thereby reducing blood glucose levels.

(5) Histological Analysis of Liver Tissue

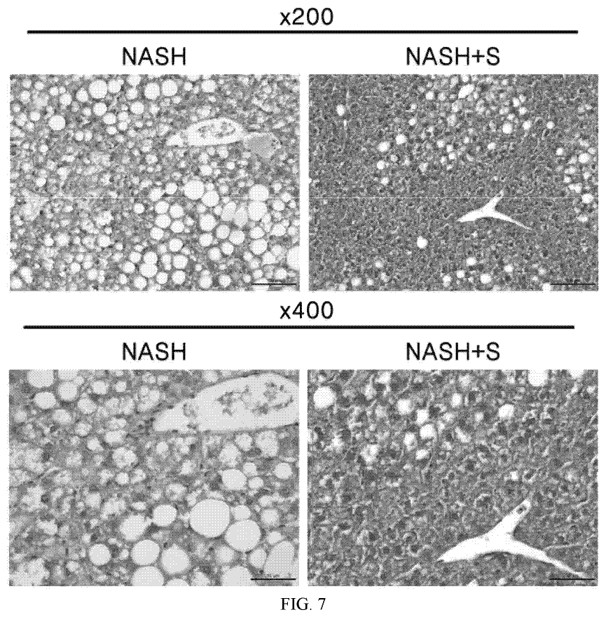

After completing the above experiment on each mouse of the control group and the sericin-treated group, extraction of liver tissue was performed under isoflurane inhalation anesthesia, followed by hematoxylin and eosin (H and E) staining. Specifically, the nuclei of liver cells were first stained with a Harris hematoxylin staining solution for 30 seconds, followed by cytoplasmic staining with an eosin solution. Results are shown in FIG. 7.

From the results shown in FIG. 7, it can be seen that the sericin-treated group showed reduction in hepatic fat accumulation and hepatic inflammatory infiltration, as compared with the control group.

(6) Biochemical Analysis

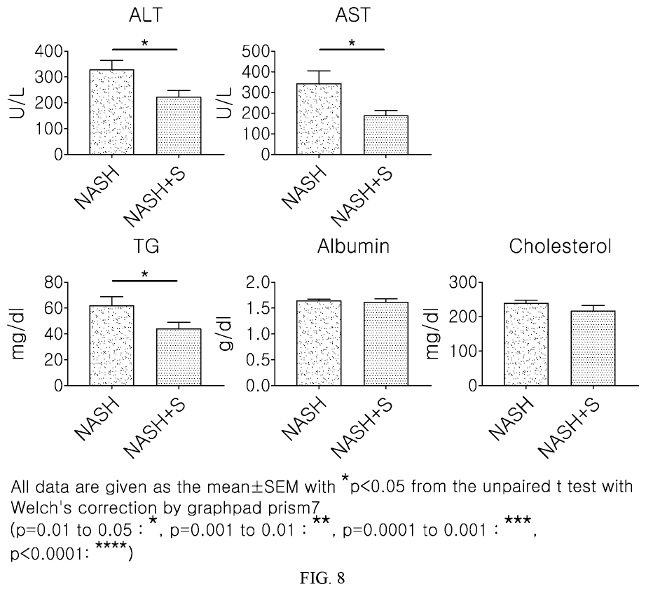

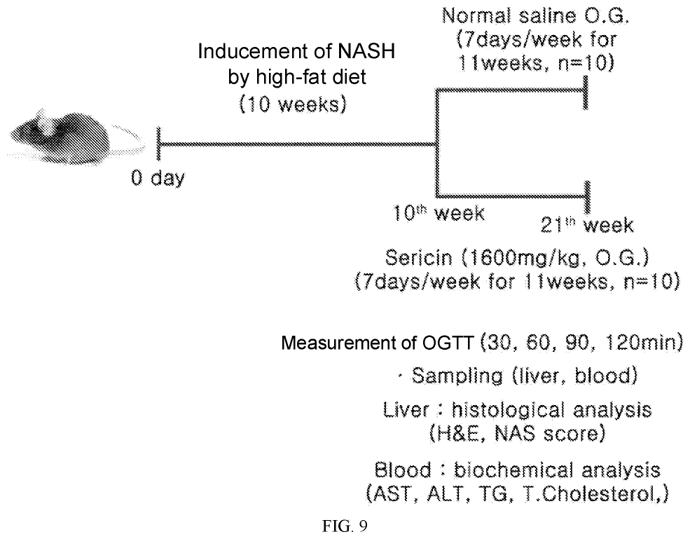

After completing the above experiment on each mouse of the control and the sericin-treated group and before extraction of liver tissue, the levels of ALT, AST, triglyceride (TG), albumin, and cholesterol in the blood were measured. Results are shown in FIG. 8.

The results show that ALT, AST, and TG levels were significantly lower in the sericin-treated group than in the control group and there was no difference in albumin and cholesterol levels between the sericin-treated group and the control group.

1. A composition for treatment, prevention or alleviation of fatty liver diseases, wherein the composition comprising sericin as an active ingredient.

2. The composition according to claim 1, wherein the fatty liver diseases are alcoholic fatty liver or non-alcoholic fatty liver.

3. The composition according to claim 1, wherein the sericin has a molecular weight distribution in a range of 200 Da to 400 kDa.

4. The composition according to claim 3, wherein the sericin has a molecular weight distribution having two main peaks located between 200 Da to 400 kDa.

5. The composition according to claim 4, wherein the sericin has a molecular weight distribution having a first main peak located between 1,000 Da and 1,700 Da and a second main peak located between 10 kDa to 30 kDa.

6. The composition according to claim 1, wherein the sericin is present in an amount of 0.01 wt % to 90 wt % based on the total weight of the composition.

7. The composition according to claim 1, wherein the composition is a pharmaceutical or food composition.

8. The composition according to claim 7, further comprising:
a pharmaceutically acceptable excipient or a sitologically acceptable excipient.

9. The composition according to claim 1, wherein the composition further has a function of reducing blood glucose levels.

10. A method of preparing a composition for treatment, prevention, or alleviation of fatty liver, the composition comprising sericin and a pharmaceutically or sitologically acceptable excipient, the method comprising:
mixing the sericin with the pharmaceutically or sitologically acceptable excipient.

11. The method according to claim 10, wherein the excipient comprises at least one selected from the group of fillers, an extender, a binder, a wetting agent, a flavoring agent, a preservative, a sweetener, a disintegrant, a surfactant, a carrier, or a diluent.

12. The method according to claim 10, wherein the fatty liver is alcoholic fatty liver or non-alcoholic fatty liver.

13. The method according to claim 10, wherein the sericin has a molecular weight distribution having two main peaks located between 200 Da and 400 kDa.

14. The method according to claim 13, wherein the sericin has a molecular weight distribution having a first main peak located between 1,000 Da and 1,700 Da and a second main peak located between 10 kDa and 30 kDa.
